(19) United States
(12) Patent Application Publication
(10) Pub. No.: US 2006/0035267 A1
(43) Pub. Date: Feb. 16, 2006

(54) Optimal polyvalent vaccine for cancer

(76) Inventors: Philip O. Livingston, New York, NY (US); Govindaswami Ragupathi, New York, NY (US)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/246,752

(22) Filed: Oct. 7, 2005

Related U.S. Application Data
(63) Continuation-in-part of application No. PCT/US04/11122, filed on Apr. 9, 2004.
(60) Provisional application No. 60/461,622, filed on Apr. 9, 2003.

(51) Int. Cl. C12Q 1/68; G01N 33/574; A61K 39/00

(52) U.S. Cl. 435/6; 435/7.23; 424/185.1

(57) ABSTRACT

This invention provides a method for identification of the optimal combination of a polyvalent vaccine against a cancer comprising steps of: a) selection of an appropriate cancer cell line; and b) detection of the expression of antigens on the surface of said cell line of the cancer, wherein the antigens expressed will be used in the polyvalent vaccine. This invention also provides a method for identification of the optimal combination of a polyvalent vaccine against a cancer comprising steps of: a) selection of an appropriate cancer cell line and b) detection of the immunogenicity will be used in the polyvalent vaccine. This invention provides various uses of the identified polyvalent vaccine.

This application is a Continuation-In-Part of International Application No. PCT/US2004/011122, filed Apr. 9, 2004, which claims the benefit of U.S. Ser. No. 60/461,622, filed Apr. 09, 2003. The disclosures of the preceding applications are hereby incorporated in their entireties by reference into this application.

This application was supported in part by NIH Grant No. P01CA33049. Accordingly, the United States Government may have certain rights in this invention.

Throughout this application, various references are cited. Disclosures of these references are hereby incorporated by reference in their entireties into this application to more fully describe the state of the art to which this invention pertains.

### BACKGROUND OF THE INVENTION

Tumor-specific antigens have been identified and pursued as targets for vaccines. In patients with small cell lung cancer (SCLC), vaccination with a SCLC specific tumor antigen conjugated to Keyhole Limpet Hemocyanin (KLH) resulted in high titer antibody response (15). Inclusion of tumor-specific antigen(s) in a polyvalent vaccine for inducing antibody-mediated immune response was described in WO2003003985.

It is an object of this invention to select the lowest number of antigens for inclusion in a vaccine that would cover essentially all tumors of a given type. It was important to select the smallest number of antigens that are needed for maximal effect. Too few antigens and some patients' tumors would not express enough of the included antigens to regress in the presence of even high titers of antibodies against each antigen. Too many antigens and vaccine production becomes much more expensive and difficult.

Therefore, there is a need for a method for determining the antigens, or combinations thereof, expressed on a tumor cell of interest which are capable of producing the optimal antibody response for inclusion in a polyvalent conjugate vaccine.

### SUMMARY OF THE INVENTION

The invention disclosed herein provides a general methodology to determine the optimal combination of a single polyvalent vaccine against different cancers. This invention provides a system which would identify the optimal combination.

This invention also provides a method for identification of the optimal combination of a polyvalent vaccine against a cancer comprising steps of: a) selection of a cancer cell line; and b) detection of the expression of antigens on the surface of said cell line of the cancer, wherein the antigens expressed will be used in the polyvalent vaccine.

This invention further provides a method for identification of the optimal combination of a polyvalent vaccine against a cancer comprising steps of: a) selection of an appropriate cancer cell line and b) detection of the immunogenicity of antigens on the surface of said cell line, wherein the antigens showing said immunogenicity will be used in the polyvalent vaccine.

This invention provides an optimal combination of a polyvalent vaccine against cancer. In an embodiment this invention provides a tetravalent vaccine for small cell lung cancer targeting GM2, Fucosyl GM1, Globo H and polysialic acid. The antigens conjugated to a carrier, such as keyhole limpet hemocyanin, to form the tetravalent vaccine for SCLC are GM2, Fucosyl GM1, Globo H and N-propionylated polysialic acid.

This invention provides a vaccine for targeting tumor specific antigens expressed on a tumor cell of interest to produce tumor cell cytotoxicity, prepared according to the process comprising the steps of: (1) identifying antigens most widely expressed on the tumor cell; (2) selecting a combination of the antigens identified in step (1) which achieves optimal antibody-mediated immune response against the tumor cell, wherein a first antibody against one antigen does not inhibit a second antibody against another antigen; and (3) conjugating the antigens selected in step (2) to a carrier to form the vaccine.

This invention provides a vaccine for targeting tumor specific antigens expressed on a tumor cell of interest to produce tumor cell cytotoxicity, prepared according to the process comprising the steps of: (1) identifying antigens most widely expressed on the tumor cell; (2) selecting a combination of the antigens identified in step (1) which achieves optimal antibody-mediated immune response against the tumor cell with a minimum number of antigens, wherein a first antibody against one antigen does not inhibit a second antibody against another antigen; and (3) conjugating the antigens selected in step (2) to a carrier to form the vaccine.

This invention provides a method of treating small cell lung cancer, comprising administering an effective amount of the vaccine of the invention to a subject, wherein the antigens conjugated to the carrier are GM2, fucosyl GM1, globo H and N-propionylated polysialic acid, and wherein the carrier is keyhole limpet hemocyanin.

Finally, this invention provides a composition for treating small cell lung cancer, said composition comprising an effective amount of antigens comprising GM2, fucosyl GM1, globo H and N-propionylated polysialic acid, wherein the antigens are conjugated to keyhole limpet hemocyanin, wherein an antibody against one antigen does not inhibit other antibodies against other antigens, and wherein antibodies against the antigens have high cell surface reactivity.

### DETAILED DESCRIPTION OF THE FIGURES

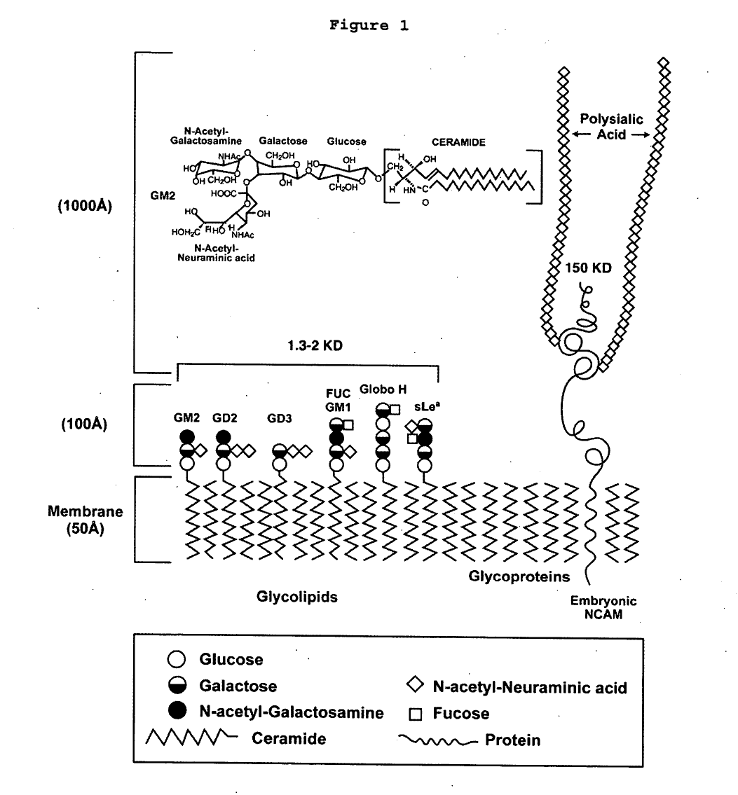

FIG. 1. Glycolipid and glycoprotein antigens expressed at the SCLC cell surface.

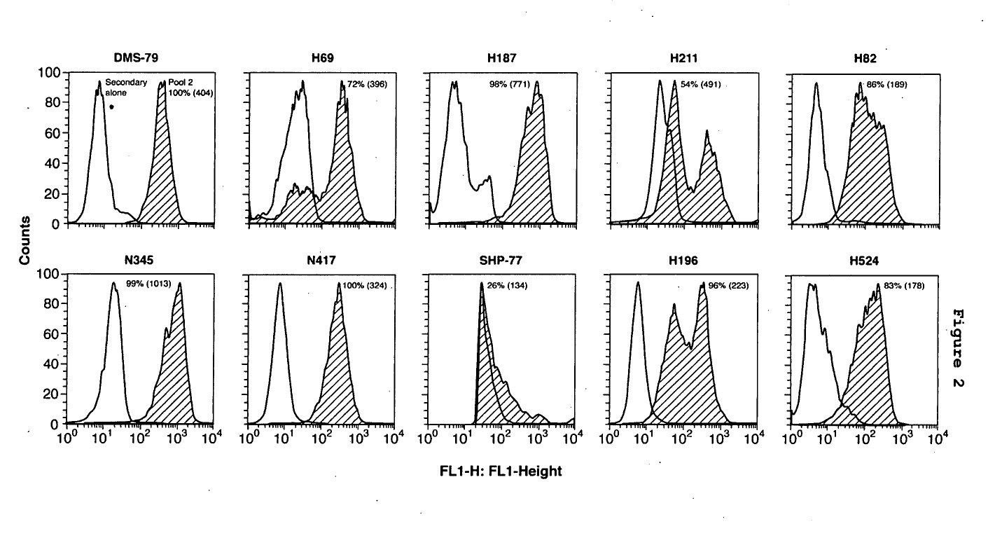

FIG. 2. IgMFACS results against 10 SCLC cell lines with the 4 mAb pool (Pool 2) containing PGNX (GM2), F12 (fucosyl GM1), VK9 (globo H) and 5A5 (polysialic acid). Peaks represent result with anti-human IgM secondary antibody alone or with the four mAb Pool 2 combination. Percent position cells and (MFI) for Pool 2 are indicated.

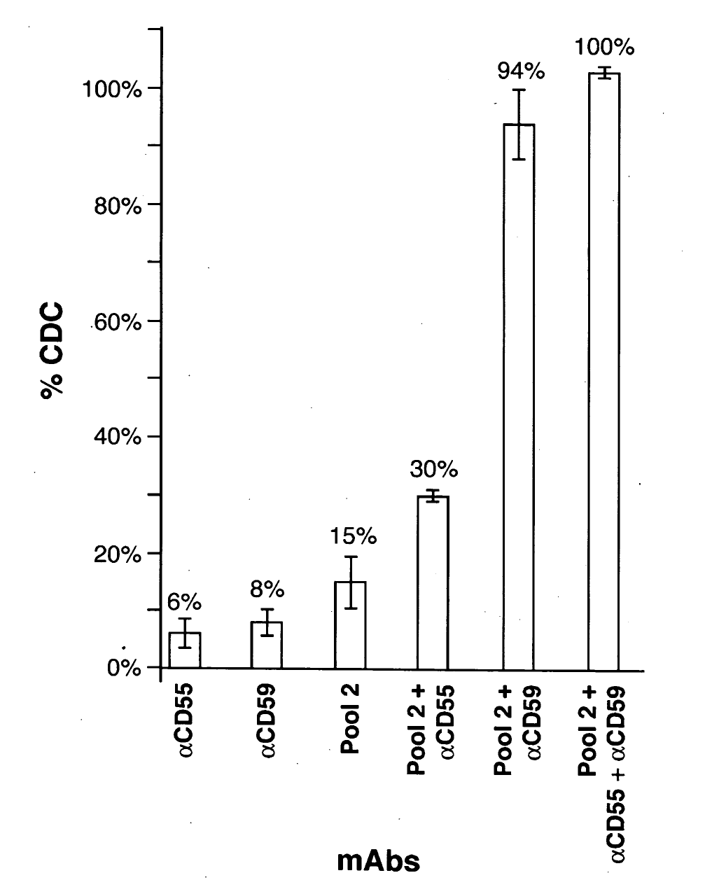

FIG. 3. Anti-CD59 mAb greatly increases CDC of SCLC cell line H345 mediated by Pool 2 (containing PGNX (GM2), F12 (fucosyl GM1), VK9 (globoH) and 5A5 (polysialic acid)). This experiment was repeated once and results of both experiments combined. Means with standard deviation are indicated. Comparison of Pool 2 alone to Pool 2 plus anti-CD59 mAb for each experiment and for the combination using the two-sample ranks test, P<0.005.

The present invention will be described in connection with preferred embodiments, however, it will be understood that this is no intent to limit the invention to the embodiments described. On the contrary, the intent is to cover all alternatives, modifications, and equivalents as may be included within the spirit and scope of the invention as defined by the appended claims.

### DETAILED DESCRIPTION OF THE INVENION

The invention disclosed herein provides a general methodology to determine the optimal combination of antigens for polyvalent vaccines against different cancers. In the literature, many antigens have been described as being expressed on the surface of cancerous cells. In designing which antigens should be used for vaccine, this invention provides a system which would identify the optimal combination.

This invention also provides a method for identification of the optimal combination of a polyvalent vaccine against a cancer comprising steps of: a) selection of an appropriate cancer cell line; and b) detection of the expression of antigens on the surface of said cell line of the cancer, wherein the antigens expressed will be used in the polyvalent vaccine.

International Patent Application No. PCT/US02/21348 (International Publication No. WO 03/003985 A2, Jan. 16, 2003) discloses a polyvalent vaccine comprising at least two conjugated antigens selected from a group containing glycolipid antigen, polysaccharide antigen, mucin antigen, glycosylated mucin antigen and an appropriate adjuvant. PCT/US02/21348 also provides a multivalent vaccine comprising at least two of the following: glycosylated MUC-1-32 mer, Globo H, GM2, Le^{γ}, Tn(c), sTN(c), and TF(c).

The current invention provides an in vitro system which predicts and optimizes the combination of said vaccine.

In an embodiment, more than one cancerous cell line is used for said identification of the optimal confirmation of a polyvalent vaccine. In another embodiment, the expression of the antigens is detected by specific antibody. In a further embodiment, the antibody is a monoclonal antibody. In a separate embodiment, the expression is detected by Fluorescence Activated Cell Sorter (FACS).

This invention further provides a method for identification of the optimal combination of a polyvalent vaccine against a cancer comprising steps of: a) selection of an appropriate cancer cell line and b) detection of the immunogenicity of antigens on the surface of said cell line, wherein the antigens showing said immunogenicity will be used in the polyvalent vaccine.

As used herein, immunogenicity describes the quality of a substance which is able to provoke an immune response against the substance, a measure of how able the substance is at provoking an immune response against it. This response includes cell-mediated and humoral responses.

In an embodiment, the immunogenicity of antigens is determined by the Complement Dependent Cytotoxicity assay. In another embodiment, the cancer is a small cell lung cancer.

This invention further provides the optimal combination identification by the above methods.

This invention also provides an effective amount of a. polyvalent vaccine for small cell lung cancer targeting GM2, Fucosyl GM1, Globo H and polysialic acid.

In an embodiment, the antigens are conjugated. In a further embodiment, the antigens are conjugated to Keyhole Limpet Hemocyanin.

In yet another embodiment, the above vaccine includes an appropriate adjuvant. The appropriate adjuvant should be able to booster the immunogenicity of the vaccine. In a further embodiment, the adjuvant is saponin-based adjuvant.

The saponin-based adjuvants include but are not limited to QS21 and GPI-0100.

This invention provides a vaccine for targeting tumor specific antigens expressed on a tumor cell of interest to produce tumor cell cytotoxicity, prepared according to the process comprising the steps of: (1) identifying antigens most widely expressed on the tumor cell; (2) selecting a combination of the antigens identified in step (1) which achieves optimal antibody-mediated immune response against the tumor cell, wherein a first antibody against one antigen does not inhibit a second antibody against another antigen; and (3) conjugating the antigens selected in step (2) to a carrier to form the vaccine. In an embodiment, the selection step (2) above further comprises pooling the antigens into one or more combinations, measuring the antibody-mediated immune response produced by each combination, and selecting the combination capable of achieving the strongest antibody-mediated immune response.

This invention provides a vaccine for targeting tumor specific antigens expressed on a tumor cell of interest to produce tumor cell cytotoxicity, prepared according to the process comprising the steps of: (1) identifying antigens most widely expressed on the tumor cell; (2) selecting a combination of the antigens identified in step (1) which achieves optimal antibody-mediated immune response against the tumor cell with a minimum number of antigens, wherein a first antibody against one antigen does not inhibit a second antibody against another antigen; and (3) conjugating the antigens selected in step (2) to a carrier to form the vaccine. In an embodiment, the selection step (2) above further comprises pooling the antigens into one or more combinations, measuring the antibody-mediated immune response produced by each combination, and selecting the combination capable of achieving the strongest antibody-mediated immune response with a minimum number of antigens.

As used herein, “Optimal antibody-mediated immune response” means, for example, maximum anti-tumor cytotoxic effect. As used herein, “a minimum number of antigens” means, for example, the lowest possible number of antigens necessary for a polyvalent vaccine to achieve maximum anti-tumor cytotoxic effect. For example, a combination of four antigens, i.e., GM2, fucosyl GM1, globo H and N-propionylated polysialic acid, conjugated to KLH is sufficient to achieve maximum anti-tumor cytotoxicity against SCLC.

In an embodiment, the antibody-mediated immune response is determined by cell surface reactivity of the antibody against the antigen. In another embodiment, the carrier is an immune modulator. In a further embodiment, the tumor cell is obtained from biopsy specimen. In a further embodiment, the antigens are identified using a specific antibody or a monoclonal antibody. In a further embodiment, tumor cell is small cell lung cancer cell. In a further embodiment, the antigens conjugated to a carrier are GM2, fucosyl GM1, globo H and N-propionylated polysialic acid. In a further embodiment, the antigens are conjugated to keyhole limpet hemocyanin. In a further embodiment, the vaccine of the invention further comprises an adjuvant including, but not limited to, QS-21 or GPI-0100.

This invention provides a method of treating small cell lung cancer, comprising administering an effective amount of the vaccine of the invention to a subject, wherein the antigens conjugated to the carrier are GM2, fucosyl GM1, globo H and N-propionylated polysialic acid, and wherein the carrier is keyhole limpet hemocyanin. In an embodiment, the vaccine is administered with an adjuvant including, but is not limited to, QS-21 or GPI-0100. In another embodiment, the adjuvant is administered at the same site as the vaccine of the invention.

In a further embodiment, the vaccine of the invention is administered intramuscularly or subcutaneously. In a further embodiment, the vaccine comprises 1 to 50 mcg of each antigen. In a further embodiment, the vaccine comprises 10-30 mcg each of GM2, fucosyl GM1 and Globo H and 3-10 mcg of N-propionylated polysialic acid. In a further embodiment, the vaccine comprises 1 mcg of N-propionylated polysialic acid and 3 mcg of fucosyl GM1. The dosages mentioned do not include the weight of the carrier.

This invention provides a composition for treating small cell lung cancer, said composition comprising an effective amount of antigens comprising GM2, fucosyl GM1, globo H and N-propionylated polysialic acid, wherein the antigens are conjugated to keyhole limpet hemocyanin, wherein an antibody against one antigen does not inhibit other antibodies against other antigens, and wherein antibodies against the antigens have high cell surface reactivity. In an embodiment, the composition further comprises an adjuvant including, but is not limited to, QS-21 or GPI-0100.

The invention will be better understood by reference to the Experimental Details which follow, but those skilled in the art will readily appreciate that the specific experiments detailed are only illustrative, and are not meant to limit the invention as described herein, which is defined by the claims which follow thereafter.

Tetravalent Vaccine Optimized for Small Cell Lung Cancer

Small cell lung cancer (SCLC) biopsy specimens previously have been screened with monoclonal antibodies (mAb) against thirty potential target antigens to identify those that are most widely expressed, i.e., on >50% of cancer cells in >60% of biopsy specimens (30-32). The glycolipids GM2, fucosyl GM1, sLea and globo H, and polysialic acid (polySA) on embryonal NCAM filled these criteria. Two additional glycolipids, GD2 and GD3, have been described by others to also be prevalent on SCLC (2, 5) and a multicenter randomized Phase 3 trial with an anti-idiotype vaccine targeting GD3 (4, 9) has recently been completed. These are all cell surface antigens that were demonstrated to be consistently immunogenic in patients when conjugated to Keyhole Limpet Hemocyanin (KLH) and mixed with immunological adjuvant QS-21 (10, 26, 8, 25, 24, 15, 18, 29) (excepting sialyl Lewis^{a} (sLe^{a}) which has not been tested) . They are all excellent candidates for inclusion in a polyvalent, antibody-inducing vaccine against SCLC.

GM2, Fucosyl GM1, Globo H and polySA were the most widespread of the SCLC cell surface antigens in the initial screen using immunohistochemistry with biopsy specimens. These four antigens were the first choices for incorporation into a polyvalent vaccine against SCLC cell surface. Prior to preparing this tetravalent conjugate vaccine, experiments were performed to confirm that mixtures of antibodies against these antigens result in stronger cell surface reactivity than any individual antibodies and to determine whether inclusion of additional antigens would yield higher cell-surface reactivity against SCLC. Initially, there were two relevant concerns. First, that the SCLC cell lines would prove resistant to complement activation and complement dependent cytotoxicity (CDC), suggesting SCLC in patients would be resistant to complement targeting and cytotoxicity. Second, that antibodies against polySA which may be a poor target for CDC as a consequence of the great distance it extends from the cell surface (15), would block CDC mediated by mAbs against other antigens. 10 SCLC cell lines were tested by flow cytometry and complement dependent cytotoxicity (CDC), with monoclonal antibodies against these seven target antigens individually or pooled in different combinations.

### Experimental Details

The invention being generally described, will be more readily understood by reference to the following examples which are included merely for purposes of illustration of certain aspects and embodiments of the present invention, and are not intended to limit the invention.

Cell lines: All SCLC cell lines were purchased from the American Type Culture Collection (ATCC). (Manassas, Va.). The cell lines are listed in Tables 1 and 2. The origin of each is listed by the ATCC as SCLC, obtained from biopsy of lung nodules except for H82, H187 and H196 which originated from pleural effusions and H211 and H345 which originated from bone marrow biopsies. SHP77 is listed as large cell variant SCLC.

Monoclonal antibodies (mAbs): The target antigens for the seven mAbs, the source of the mabs and the concentration used in the FACS studies are described below. GM2, mAb PGNX, Progenics Pharmaceuticals Inc. (Tarrytown, N.Y.), ascites 0.5 μl/ml.

Fucosyl GM1, mAb F12, Dr. Thomas Brezicka (Goteborg, Sweden), 0.1 μg/ml.

Globo H, mAb VK9, Kenneth Lloyd (MSKCC), 20 μg/ml. Polysialic acid, mAb 5A5, Urs Rutishauser (MSKCC), ascites 0.1 μg/ml.

GD2, mAb 3F8, Dr. Nai-Kong Cheung (MSKCC), 0.4 μg/ml.

GD3, mAb R24, Dr. Paul Chapman (MSKCC), 0.4 μg/ml. sLe^{a}, mAb 19.9, purchased from Signet (Dedham, Mass.), supernatant 0.05 μl/ml.

These mAbs, concentrations and mAb subclasses are listed in Table 1. The antigens recognized by these mAbs are shown in FIG. 1.

Fluorescence Activated Cell Sorter (FACS) Assay: The ten SCLC cell lines served as targets. Single cell suspensions of 2×10⁵ cells/tube were washed with 3% fetal calf serum in PBS and incubated with 20 μl of diluted test mAb for 30 min on ice. The final concentrations of each mAb in mAb pools 1-5 are the same as when mAbs were tested singly. MAbs were tested on each of the ten cell lines over at least a 1,000 fold range of concentrations, generally at final concentrations between 1 μg/ml (or 10 μl/ml) and 0.001 ug/ml (or 0.01 μl/ml). Percent positive cells and mean fluorescent intensity (MFI) generally peaked and then plateaued for each cell line as concentrations increased. The lowest concentration giving peak MFI was determined for each cell line. The concentration giving an MFI that was 25% of the peak MFI in the majority of positive cell lines was selected. In most cases this approximated the percent positive cells and MFI achievable with sera from patients vaccinated with these antigens conjugated to KLH (though on other cell lines) (8, 10, 15, 18, 24-26, 29). After washing the cells twice with 3% FCS in PBS, 20 μl of 1:25 goat anti-mouse IgG or IgM-labeled with FITC was added. The suspension was mixed, incubated for 30 min and washed. The percent positive population and mean fluorescence intensity of stained cells were analyzed using a FACS Scan (Becton-Dickinson, Calif.) (8, 25) with percent positive cells for second antibody alone gaited at 1%.

Complement Dependent Cytotoxicity (CDC) and Antibody Dependent Cellular Cytotoxicity (ADCC): Complement dependent cytotoxicity was assayed on the ten cell lines using a 2-hour 51 chromium release assay as previously described (24) with human complement and single mabs or mAb pools at the concentrations indicated in Table 2. The final concentrations of each mAb in mAb pools 1-5 are the same as when mAbs were tested singly. Though the concentration of mabs in CDC assays was generally higher than in FACS assays, the level of CDC was comparable to that achieved using sera from some patients vaccinated with fucosyl GM1 and tested against DMS79 (8, 15), or with GM2 or globo H and tested against other cell lines (18, 29). Approximately 10⁷ cells were labeled with 100 μCi of Na₂ ⁵¹CrO₄ (New England Nuclear, Boston, Mass.) in 1% HSA for 2 h at 37° C., shaking every 15 min. The cells were washed four times and brought to a concentration of 2×10⁶ live cells/ml. Fifty microliters of labeled cells were mixed with 50 μl of undiluted mAb or with medium alone in 96-well, round-bottomed plates (Corning, New York, N.Y.) and incubated at 4° C. on a shaker for 45 min. Human complement (Sigma Diagnostics, St. Louis, Mo.) diluted 1:5 with 1% HSA was added, at 100 μl/well, and incubated at 37° C. for 2 h. The plates were spun at 1000 g for 3 min, and an aliquot of 30 μl of supernatant from each well was read by a gamma counter to determine the amount of ⁵¹Cr released. All samples were performed in triplicate and included control wells for maximum release and for spontaneous release in the absence of complement.

Spontaneous release (the amount released by target cells incubated with complement alone) was subtracted from both experimental and maximal release values. Maximum release was the amount of radioactivity released by target cells after a 2-hour incubation with 1% Triton X-100. Percent specific release (CDC) was calculated as corrected experimental/corrected maximal release. Where indicated, concentrations of anti-CD55 and anti-CD59 between 25 and 150 μg/ml were added to CDC assay wells with the mabs or mAb pools to counteract inhibition mediated by CD55 and CD59. MAb clone BRIC 216 against CD55 and mAb MEM-43 against CD59 were purchased from Serotec Inc. (Raleigh, N.C.)

Cell Surface Reactivity Demonstrated by FACS

Cell surface reactivity for the 7 monoclonal antibodies utilized at the concentrations summarized in Table 1 ranged from 1% to more than 99% in the 10 SCLC cell lines. Two of the mAbs (PGNX recognizing GM2 and 5A5 recognizing polySA) resulted in 50% or more positive cells in 6 of the 10 SCLC cell lines. The other mAbs demonstrated comparable reactivity with 5 or fewer cell lines. On the other hand, when the mAbs were pooled in different combinations using the same mAb concentration, 9 of 10 cell lines demonstrated 50% or greater positive cells.

Combination containing mabs against fucosyl GM1, GM2, globoH and polysialic acid (the four mAb pool) was optimal, the addition of antibodies against GD2, GD3 and sialyl LewisA had little additional impact. While some cell lines such as DMS79 and H187 were strongly positive with 6 of the 7 mAbs, others such as SHP77, H211 and H82 or H196 were positive with only zero to two of the mAbs. However, when the antibodies were pooled in different combinations only SHP77 continued to demonstrate fewer than 50% positive cells. Cell surface reactivity by FACS for the 10 cell lines with the 4 mAb pool is demonstrated in greater detail in FIG. 2. With the exception of cell line SHP77, strong cell surface reactivity was demonstrated against all cell lines.

TABLE 1
Reactivitv of single mAbs and pools of mAbs against cell surface antigens on ten SCLC cell lines

| | | | | | DMS-79 | H69 | H187 | H345 | N417 | SHP77 | H211 | H82 | H524 | H196 |
|---|---|---|---|---|---|---|---|---|---|---|---|---|---|---|
| Antigen | Monoclonal AB | Class/Subclass | Concentration | | % (+) | % (+) | % (+) | % (+) | % (+) | % (+) | % (+) | % (+) | % (+) | % (+) |
| FucoGm1 | F12 | IgG3 | 0.1 | ug/ml | 96% | 21% | 66% | 12% | 1% | 1% | 10% | 1% | 1% | 1% |
| GD2 | 3F8 | IgG3 | .4 | ug/ml | 20% | 68% | 66% | 38% | 12% | 1% | 3% | 58% | 98% | 37% |
| GD3 | R24 | IgG3 | .4 | ug/ml | 12% | 70% | 30% | 39% | 2% | 2% | 4% | 42% | 75% | 2% |
| GM2 | PGNX - ascites | IgM | 0.5 | ul/ml | 54% | 13% | 90% | 1% | 99% | 25% | 27% | 86% | 90% | 81% |
| PolySA | 5A5 - ascites | IgM | 0.1 | ul/ml | 99% | 58% | 90% | 99% | 88% | 46% | 25% | 15% | 86% | 2% |
| GloboH | VK9 | IgG3 | 20 | ug/ml | 96% | 30% | 99% | 10% | 41% | 6% | 34% | 10% | 12% | 68% |
| Sialyl LeA | CA19.9 - supe | IgG1 | 0.05 | ul supe | 97% | 88% | 4% | 14% | 1% | 5% | 1% | 1% | 1% | 1% |

| | | | | | | | | | | |
|---|---|---|---|---|---|---|---|---|---|---|
| Pool 1 = FucoGM1, | 99% | 44% | 99% | 25% | 99% | 25% | 54% | 74% | 59% | 96% |
| GM2, GloboH | | | | | | | | | | |

| | | | | | | | | | | |
|---|---|---|---|---|---|---|---|---|---|---|
| Pool 2 = FucoGM1, GM2, | 100% | 50% | 98% | 99% | 100% | 26% | 54% | 86% | 83% | 96% |
| GloboH, PolySA | | | | | | | | | | |
| Pool 3 = FucGM1, GM2, GloboH, | 99% | 52% | 99% | 98% | 99% | 46% | 44% | 90% | 90% | 98% |
| PolySA, Sialyl LeA | | | | | | | | | | |
| Pool 4 = FucoGM1, GM2, GloboH, | 99% | 67% | 99% | 99% | 99% | 25% | 40% | 91% | 96% | 94% |
| GD2, GD3, PolySA | | | | | | | | | | |

| | | | | | | | | | | |
|---|---|---|---|---|---|---|---|---|---|---|
| Pool 5 = FucoGM1, GM2, GloboH, GD2, GD3, PolySA, Sialyl LeA | 99% | 79% | 99% | 99% | 99% | 35% | 50% | 92% | 98% | 94% |

Cell Surface Reactivity Demonstrated by CDC

Complement dependent cytotoxicity (CDC) assays using human complement demonstrated 30% or greater lysis in 5 of the 10 cell lines with PGNX against GM2, in 3-4 of the 10 cell lines with mAbs against fucosylated GM1, GD2 and GD3, and none of the cell lines with mAb against polysialic acid, globoH and sialyl Le^{A} (see Table 2). The 4 antibody pool including fucosylated GM1, GM2, globoH and polysialic acid resulted in greater than 30% cytotoxicity for 9 of the 10 cell lines. This was increased slightly by the addition of antibodies against GD2 and GD3 but still one cell line, H345, had less than 30% cytotoxicity despite the fact that 99% of the H345 cells had strong reactivity by FACS with the same pools. Aside from H345, FACS and CDC correlated fairly closely, with some such as HSP77 and H211 demonstrating stronger than expected CDC.

TABLE 2
Complement dependent cvtotoxicitv of single mAbs and pools of
mAbs against cell surface antigens on ten SCLC cell lines

| | | | | | DMS-79 | H69 | H187 | H345 | N417 | SHP77 | H211 | H82 | H524 | H196 |
|---|---|---|---|---|---|---|---|---|---|---|---|---|---|---|
| | | | | | % | % | % | % | % | % | % | % | % | % |
| Antigen | Monoclonal AB | Class/Subclass | Concentration | | Lysis | Lysis | Lysis | Lysis | Lysis | Lysis | Lysis | Lysis | Lysis | Lysis |
| FucoGm1 | F12 | IgG3 | 1.25 | ug/ml | 81% | 34% | 69% | 1% | 0% | 0% | 6% | 0% | 32% | 4% |
| GD2 | 3F8 | IgG3 | 1.25 | ug/ml | 10% | 16% | 3% | 4% | 59% | 0% | 0% | 43% | 62% | 3% |
| GD3 | R24 | IgG3 | 10 | ug/ml | 3% | 28% | 3% | 8% | 38% | 0% | 0% | 66% | 53% | 0% |
| GM2 | PGNX - ascites | IgM | 2.5 | ul/ml | 32% | 1% | 3% | 0% | 79% | 52% | 0% | 50% | 51% | 27% |
| PolySA | 5A5 - ascites | IgM | 2.5 | ul/ml | 6% | 0% | 0% | 1% | 9% | 0% | 0% | 2% | 11% | 3% |
| GloboH | VK9 | IgG3 | 18.75 | ug/ml | 47% | 1% | 21% | | 20% | 0% | 0% | 5% | 12% | 0% |
| Sialyl LeA | CA19.9 - supe | IgG1 | 50 | ug/ml | 1% | 1% | 6% | 0% | 25% | 17% | 0% | 0% | 0% | 3% |

| | | | | | | | | | | |
|---|---|---|---|---|---|---|---|---|---|---|
| Pool 1 = FucoGM1, GM2, GloboH | 92% | 46% | 57% | 5% | 84% | 48% | 45% | 80% | 50% | 27% |
| Pool 2 = FucoGM1, GM2, GloboH, PolySA | 93% | 51% | 47% | 4% | 93% | 62% | 68% | 81% | 55% | 33% |

| | | | | | | | | | | |
|---|---|---|---|---|---|---|---|---|---|---|
| Pool 3 = FucGM1, GM2, GloboH, PolySA, Sialyl LeA | 85% | 51% | 56% | 4% | 86% | 48% | 51% | 83% | 55% | 34% |
| Pool 4 = FucoGM1, GM2, GloboH, GD2, GD3, PolySA | 95% | 61% | 57% | 12% | 93% | 64% | 83% | 84% | 89% | 36% |
| Pool 5 = FucoGM1, GM2, GloboH, GD2, GD3, PolySA, Sialyl LeA | 84% | 61% | 52% | 7% | 100% | 54% | 64% | 86% | 95% | 32% |

Cell Surface Expression of CD55 and CD59

CD55 was strongly expressed on 3. of the 10 cell lines (SHP77, H524 and H196) and CD59 was strongly expressed on all cell lines except H211 and H82. There was no clear correlation between expression of these 2 complement resistance factors and the level of complement dependent cytotoxicity (Table 3). H345 was one of the many strongly CD59 positive cell lines but was only moderately positive for CD55. H345 may have been negative by CDC because the predominate antigen recognized by these mabs at the cell surface is polysialic acid. Nevertheless, to explore the role of CD55 and CD59 in complement lysis against this apparently complement resistant cell line the CDC assay in the presence of anti-CD55 or anti-CD59 mabs was performed (see Table 3). Neither anti-CD55 nor anti-CD59, (nor the two in combination), were able to mediate detectable complement cytotoxicity on their own against H345. CDC mediated by the four mAb pool showed no change in the presence of 100 μg per ml of anti-CD55, but increased from 15% to 94% (P<0.005) in the presence of 100 μg per ml of anti-CD59 (FIG. 3).

TABLE 3
CORRELATION OF CD55 AND CD59 EXPRESSION ON TEN SCLC CELL LINES TO FACS AND CDC REACTIVITY

| | | DMS-79 | H69 | H187 | H345 | N417 | SHP77 | H211 | H82 | H524 | H196 |
|---|---|---|---|---|---|---|---|---|---|---|---|
| mAbs | Conc | %/MFI* | %/MFI | %/MFI | %/MFI | %/MFI | %/MFI | %/MFI | %/MFI | %/MFI | %/MFI |
| Anti-CD55 | 0.1 μg | 1%/6 | 4%/17 | 46%/13 | 31%/7 | 3%/5 | 86%/25 | 1%/13 | 13%/159 | 99%/76 | 97%/46 |
| Anti-CD59 | 0.1 μg | 99%/107 | 96%/126 | 96%/82 | 100%/177 | 100%/203 | 100%/160 | 1%/13 | 19%/166 | 100%/110 | 100%/160 |
| FACS-Pool 2 | | 100 (404) | 72 (396) | 98 (771) | 99 (1,013) | 100 (324) | 26 (134) | 54 (491) | 86 (189) | 83 (178) | 96 (223) |
| FACS-Best | | 100 (598) | 79 (229) | 99 (859) | 100 (1,028) | 100 (518) | 35 (103) | 54 (506) | 90 (214) | 98 (424) | 98 (302) |
| pool | | | | | | | | | | | |
| CDC-Pool | | 93% | 51% | 47% | 4% | 93% | 62% | 68% | 81% | 55% | 33% |
| 2 (%) | | | | | | | | | | | |
| CDC-Best | | 95% | 61% | 57% | 12% | 100% | 64% | 83% | 86% | 95% | 36% |
| pool (%) | | | | | | | | | | | |

*MFI Mean fluorescence intensity

### CONCLUSION

Biopsies of SCLC demonstrate a rich array of cell carbohydrate surface antigens. Fucosyl GM1, GM2, polysialic acid, globo H, sialyl Le^{a}, GD2 and GD3 are the most widely expressed of these. These are each excellent targets for active or passive antibody mediated immunotherapy of SCLC, but no one of these antigens has been shown to be expressed on more than 70 or 80% of SCLC biopsy specimens. This is the basis for the focus on constructing a polyvalent vaccine against several of these antigens. It has been demonstrated that pools of mabs recognize multiple SCLC cell surface antigens mediate stronger cell surface reactivity than individual mAbs.

The reactivity of mAbs against 7 different cell surface antigens on a panel of 10 SCLC cell lines using flow cytometry was measured. The concentrations of the mAbs used was selected to give ELISA and FACS titers of reactivity comparable to those achieved in patients receiving KLH conjugate vaccines against these antigens (8, 10, 15, 18, 24, 25, 26). The four antigens recognized most widely by these mAbs on biopsy specimens, and now these ten cell lines, were fucosylated GM1, GM2, globoH and polysialic acid. The number of cell lines demonstrating 50% or more positive cells by FACS increased from six or fewer to 9 of the 10 cell lines when Pool 2 (containing mAbs against these four antigens) was utilized and the remaining cell line (SHP77) was positive as well, demonstrating 26% positive cells. The addition of antibodies against GD2, GD3 and sialyl Lea had little additional impact. In previous clinical trials with polySA-KLH conjugate vaccines, antibodies against polysialic acid were unable to mediate CDC. However, vaccination with N-propionylated polysialic acid (NP-polysialic acid) has been shown to result in a consistent high titer antibody response to polysialic acid (15). Therefore, fucosyl GM1, GM2, globo H, and NP-polysialic acid were selected as the antigens for inclusion in the polyvalent vaccine for SCLC.

Experiments were performed to confirm that selection of these 4 target antigens was also optimal using complement dependent cytotoxicity assay to be sure that antibody against polySA would not interfere with CDC mediated by antibodies against the other 3 antigens.

The number of cell lines demonstrating 30% or more positive cells by CDC increased from four or fewer to nine of the ten cell lines when the four mAb pool was utilized. The remaining cell line, H345, though strongly positive by FACS was completely resistant to CDC. Several mechanisms for cancer cells to evade complement dependent cytotoxicity have been described (6, 12, 27). CD55, which interferes at the level of C3 convertase, and CD59, which interferes with assembly of the membrane attack complex, are the most widely studied of the complement activation resistance factors. It has been reported that tumor cells can avoid CDC in the face of potent FACS reactivity at the cell surface when the antigens are on elongated molecules such as mucins. This was initially detected with monoclonal antibodies and vaccine induced antibodies against MUC1 (17) but more recently also against polysialic acid (15). CDC resistance is assumed to result from the great distance from the cell surface that complement activation occurs. This is similar to the resistance to CDC described for Salmonella minnesota, Salmonella monte video, Pseudomonas aeruginosa and other “smooth” bacterial strains with long lipopolysaccharide chains (19, 26). Complement activation initiates a cascade of enzyme activities resulting in binding of C3b and eventually insertion of the C5b-9 protein complement membrane attack complex (MAC) into cell membranes to form pores. Dimensions of the MAC are 100 by 150 angstroms (7). The molecular weight of the NCAM C-terminal extracellular subunit and flanking sequence are in excess of 100 KD (14, 28), making it likely that the polysialic acid portion begins 100 angstroms or more from the cell membrane. If complement activation occurs at sites more distant than 100 angstroms from the cell membrane (see FIG. 1). This polysialic chain extends away from the lipid bilayer, the negatively charged sialic acid chain repulsed by the sialic acid rich, negatively charged cancer cell surface. If complement activation occurs at sites more distant than 100 angstroms from the cell membrane, the membrane attack complex would not form or if formed would not reach the cell membrane and a number of serum proteins would quickly inactivate the forming membrane attack complex (7). C3 mediated inflammation and opsonization, however, would remain in place.

As demonstrated here, mAb5A5 (against polySA) again proved to be a highly reactive IgM antibody, resulting in potent cell surface reactivity by FACS against 6 of the 10 SCLC cell lines. However, though this IgM antibody is certainly activating complement, it was unable to mediate complement cytotoxicity against any cell line. This is consistent with our previous finding with sera from SCLC patients after vaccination (15) and with mAb 735 which is strongly reactive with polySA positive SCLC cell lines. When mAb5A5 was added to pools of other monoclonal antibodies, however, no diminution in CDC was detected, demonstrating that there was no steric or other hindrance to CDC mediated by antibodies binding to antigens that are more intimately associated with the cell surface lipid bilayer. Overall, the CDC assay gave results which were quite similar to those obtained with FACS. The number of cell lines demonstrating more than 30% cytotoxicity (in a 2 h assay) with any one mAb increased from 0-5 cell lines with single mAbs to 9 of the 10 cell lines with the pools of antibodies.

Although most cancers of the colon and stomach are known to express CD55, it was not found on either of the two SCLC biopsies described to date (12, 20) and was seen in 0/4 (6) or 29% (27) of SCLC cell lines, consistent with our findings of strong CD55 expression in 3 of 10 SCLC cell lines. CD55 was only minimally expressed in the one SCLC that was resistant to CDC. Assuming that CD55 expression on cell lines reflects expression in vivo, it seems unlikely, that CD55 mediated CDC resistance will be a major problem in the SCLC patients that will be immunized. CD59 was strongly expressed on 8 of the 10 cell lines, but there was no clear correlation between this expression and CDC. While cell line H345 (which expressed CD55 weakly and CD59 strongly) had 15% peak CDC, it demonstrated 99% positive cells by FACS. In the presence of inhibiting levels of mAbs against CD59, however, CDC increased from 15% to 94%. This demonstrates complement activation by the 4 mAb pool which was being inhibited only at the membrane attack complex level by CD59. This strongly suggests that with the four antibody pool, nine of the ten SCLC cell lines are sensitive to CDC and that all 10 SCLC cell lines tested, including even H345, should be good targets for antibody mediated effector mechanisms such as inflammation and opsonization. These results demonstrate that a polyvalent vaccine containing fucosylated GM1, GM2, globo H and polysialic acid or N-Propionylated polysialic acid is sufficient for inducing antibodies against the great majority of SCLCs, resulting in complement activation and, in most cases, complement dependent cell cytotoxicity.

Following the teaching of this invention, it is expected that a person of ordinary skill in the art would be able prepare an antibody-inducing, polyvalent vaccine with the minimum number of antigen conjugates for other types of cancers, while achieving the optimal cancer cell cytotoxicity.

### Equivalents

Those skilled in the art will recognize, or be able to ascertain using no more than routine experimentation, many equivalents to the specific embodiments of the invention described herein. Such equivalents are intended to be encompassed by the following claims.

### REFERENCES

1. Brezicka F T, et al. (1989) Immunohistological detection of fucosyl-GM1 ganglioside in human lung cancer and normal tissues with monoclonal antibodies. Cancer Res 49(5): 1300-5

2. Brezicka T, Bergman B, Olling S, Fredman P (2000) Reacitivity of monoclonal antibodies with ganglioside antigens in human small cell lung cancer tissues. Lung Can 28: 29-36

3. Caragine T A, Okada N, Frey A B, Tomlinson S A (2002) Tumor-expressed inhibitor of the early but not late complement lytic pathway enhances tumor growth in a rat model of human breast cancer. Cancer Res 62(4):1110-1115

4. Chapman P B, Livingston P O, Morrison M E, Williams L, Houghton A N (1994) Immunization of melanoma patients with anti-idiotypic monoclonal antibody BEC2 (which mimics GD3 ganglioside): pilot trials using no immunological adjuvant. Vaccine Res. 3(2):59-69

5. Cheresh D A, Rosenberg J, Mujoo K, Hirschowitz L, Reisfeld R A (1986) Biosynthesis and expression of the disialoganglioside GD2, a relevant target antigen on small cell lung carcinoma for monoclonal antibody-mediated cytolysis. Cancer Res 46(10):5112-5118

6. Cheung N K, Walter E I, Smith-Mensah W H, Ratnoff W D, Tykocinski M L, Medof M E (1988) Decay-accelerating factor protects human tumor cells from complement-mediated cytotoxicity in vitro. J Clin Invest 81(4): 1122-8

7. Colten H R, Rosen F S (1992) Complement deficiencies. Ann Rev Immunol. 10:809 834

8. Dickler M N, Ragupathi G, Liu N X, Musselli C, Martino D J, Miller V A, Kris M G, Brezicka F T, Livingston P O, Grant S C (1999) Immunogenicity of the fucosyl-GM1-keyhole limpet hemocyanin (KLH) conjugate vaccine in patients with small cell lung cancer. Can Res 5: 2773-2779

9. Giaccone G, Debruyne C, Felip E, Millward M, D'Addario G, Thiberville L, Rome L, Zatloukal P, Legrand C (2004) Phase III study of BEC2/BCG vaccination in limited disease small cell lung cancer (LD-SCLC) patients, following response to chemotherapy and thoracic irradiation (EORTC 08971, the SIL VA study). J Clin Oncol (Meeting Abstracts) 22(14_suppl):7020

10. Helling F, Zhang A, Shang A, Adluri S, Calves M, Koganty R, Longenecker B M, Oettgen H F and Livingston P O (1995) GM2-KLH conjugate vaccine: Increased immunogenicity in melanoma patients after administration with immunological adjuvant QS-21. Cancer Res. 55:2783-2788

11. Huntsberger D V, Leaverton P (1970) Statistical inference in the biomedical sciences. Allyn and Bacon Inc., Boston.

12. Jarvis G A, Li J, Hakulinen J, Brady K A, Nordling S, Dahiya R, Meri S (1997) Expression and function of the complement membrane attack complex inhibitor protectin (CD59) in human prostate cancer. Int. J. Cancer 71: 1049-1055

13. Joiner K A (1988) Complement evasion by bacteria and parasites. Annu Rev Microbiol 42: 201-30

14. Komminoth P J, et al. (1991) Polysialic acid of the neural cell adhesion molecule distinguishes small cell lung carcinoma from carcinoids. Am J Pathol 139(2): 297-304

15. Krug L M, Ragupathi G, Ng K K, Hood C, Jennings H J, Guo Z, Kris MG, Miller V, Pizzo B, Tyson L, Baez V, Livingston P O (2004) Vaccination of small cell lung cancer patients with polysialic acid or N-propionylated polysialic acid conjugated to keyhole limpet hemocyanin. Clin Cancer Res 10(3):916-923

16. Krug L M, Ragupathi G. Hood C, Kris M G, Miller V A, Allen J R, Keding S J, Danishefsky S J, Gomez J, Tyson L, Pizzo B, Baez V, Livingston P O (2004) Vaccinatin of patients with small cell lung cancer with synthetic fucosyl GM1 conjugated to Keyhole Limpet Hemocyanin (KLH). Clin Cancer Res 10:6094-6102

17. Ragupathi G, Liu N X, Musselli C, Powell S, Lloyd K, Livingston P O (2005) Antibodies against tumor cell glycolipids and proteins, but not mucins, mediate complement-dependent cytotoxicity. J Immunol 174:5706-5712

18. Livingston P O, Zhang S, Walberg L, Ragupathi G, Helling F, Fleischer M (1997) Tumor cell reactivity mediated by IgM antibodies in sera from melanoma patients vaccinated with GM2-KLH is increased by IgG antibodies. Cancer Immunol. Immunother. 43:324-330

19. Moffitt M C, Frank M M (1994) Complement resistance in microbes. Springer Semin Immunopathol 15(4): 327-44

20. Nicholson-Weller A, Burge J, Fearon D T, Weller P F, Austen K F (1982) Isolation of a human erythrocyte membrane glycoprotein with decay-accelerating activity for C3 convertases of the complement system. J. Immunol 129: 184-189

21. Nilsson O, et al. (1984) Fucosyl-GM1-A ganglioside associated with small cell lung carcinomas. Glycoconjugate J 1: 43-49

22. Ragupathi G, Cappello S, Yi S S, Canter D, Spassova M, Bornmann W G, Danishefsky S J, Livingston P O (2002) Comparison of antibody titers after immunization with monovalent or tetravalent KLH conjugate vaccines. Vaccine 20(7-8):10301038

23. Ragupathi G, Koide F, Sathyan N, Kagan E, Spassova M, Bornmann W, Gregor P, Reis C A, Clausen H, Danishefsky S J, Livingston P O (2003) A preclinical study comparing approaches for augmenting the immunogenicity of a heptavalent KLH-conjugate vaccine against epithelial cancers. Cancer lmmunollmmunother 52(10): 608-616

24. Ragupathi G. Livingston P O, Hood C, Gathuru J, Krown S E, Chapman P B, Wolchok J D, Williams L J, Oldfield R C, Hwu, W J (2003) Consistent antibodies against ganglioside GD2 induced in patients with Melanoma by a GD2 Lactone (GD2L)-KLH conjugate vaccine plus immunological adjuvant QS-21. Clin Can Res 9(14):5214-5220

25. Ragupathi G, Meyers M, Adluri S, Howard L, Yu R K, Ritter G. Livingston P O (2000) Phase I trial with GD3-lactone-KLH conjugate and immunological adjuvant QS-21 vaccine with malignant melanoma. Int. J. Can 85: 659-666

26. Ragupathi G, Slovin S, Adluri S., Sames D, Kim I J, Kim H M, Spassova M, Bornmann W, Lloyd K O, Scher H I, Livingston P O, Danishefsky S J (1999) A fully synthetic globo H carbohydrate vaccine induces a focused humoral response in prostate cancer patients: A proof of Principle. Angewandte. Chemie 38: 563-566

27. Sakuma T, Kodama K, Hara T, Eshita Y, Shibata N, Matsumoto M, Seya T, Mori Y (1993) Levels of complement regulatory molecules in lung cancer: disappearance of the D17 epitope of CD55 in small-cell carcinoma. Jpn J Cancer Res 84(7): 753-9

28. Scheidegger E P, et al. (1994) In vitro and in vivo growth of clonal sublines of human small cell lung carcinoma is modulated by polysialic acid of the neural cell adhesion molecule. Lab Invest 70(1): 95-106

29. Slovin S F, Ragupathi G, Adluri S, Ungers G, Terry K, Kim S, Spassova M, Bornmann W G, Fazzari M, Dantis L, Olkiewicz K, Lloyd K O, Livingston P O, Danishefsky S J, Scher H I (1999) Carbohydrate vaccines in cancer: immunogenicity of a fully synthetic globo H hexasaccharide conjugate in man. Proc Natl Acad Sci 96:5710-5715

30. Zhang S, Cordon-Cardo C, Zhang H S, Reuter V E, Adluri S, Hamilton W B, Lloyd K W, Livingston P O (1997) Selection of carbohydrate tumor antigens as targets for immune attack using immunohistochemistry. I. Focus on Gangliosides. Int. J. Cancer 73:42-49

31. Zhang S, Zhang H S, Cordon-Cardo C, Ragupathi G, Livingston P O (1998) Selection of tumor antigens as targets for immune attack using immunohistochemistry: III protein antigens. Clin. Cancer Res. 4: 2669-2676

32. Zhang S, Zhang H S, Cordon-Cardo C, Reuter V E, Singhal, A K, Lloyd K O, Livingston P O (1997) Selection of tumor antigens as targets for immune attack using immunohistochemistry. II. Blood group-related antigens. Int. J. Cancer 73: 50-56

33. Souhami R L, et al. (1991) Antigens of lung cancer: results of the second international workshop on lung cancer antigens. J Natl Cancer Inst 83(9): 609-12

34. Liu N X, Ragupathi G, Cappello S, Musselli C, Lloyd K O, Livingston P O (1993) Antibodies against tumor glycolipids and proteins, but not mucins, mediate complement-dependent cytotoxicity (CDC). Proceedings AACR 41: #5582

36. Price M R, Tendler S J B (1993) Polymorphic epithelial mucins (PEM): molecular characteristics and association with breast cancer. The Breast 2: 3-7

37. Livingston P O, et al. (2005) Selection of GM2, fucosyl GM1, globo H and polysialic acid as targets on small cell lung cancers for antibody mediated immunotherapy. Cancer Immunol. Immunother 54: 1018-1025

1-13. (canceled)

14. A vaccine for targeting tumor specific antigens expressed on a tumor cell of interest to produce tumor cell cytotoxicity, prepared according to the process comprising the steps of:
(1) identifying antigens most widely expressed on the tumor cell;
(2) selecting a combination of the antigens identified in step (1) which achieves optimal antibody-mediated immune response against the tumor cell, wherein a first antibody against one antigen does not inhibit a second antibody against another antigen; and
(3) conjugating the antigens selected in step (2) to a carrier to form the vaccine.

15. A vaccine for targeting tumor specific antigens expressed on a tumor cell of interest to produce tumor cell cytotoxicity, prepared according to the process comprising the steps of:
(1) identifying antigens most widely expressed on the tumor cell;
(2) selecting a combination of the antigens identified in step (1) which achieves optimal antibody-mediated immune response against the tumor cell with a minimum number of antigens, wherein a first antibody against one antigen does not inhibit a second antibody against another antigen; and
(3) conjugating the antigens selected in step (2) to a carrier to form the vaccine.

16. The vaccine of claim 14, wherein the selecting step (2) further comprises pooling the antigens into one or more combinations, measuring the antibody-mediated immune response produced by each combination, and selecting the combination capable of achieving the strongest antibody-mediated immune response.

17. The vaccine of claim 15, wherein the selecting step (2) further comprises pooling the antigens into one or more combinations, measuring the antibody-mediated immune response produced by each combination, and selecting the combination capable of achieving the strongest antibody-mediated immune response with a minimum number of antigens.

18. The vaccine claims 16, wherein the antibody-mediated immune response is determined by cell surface reactivity of the antibody against the antigen.

19. The vaccine of claim 14, wherein the carrier is an immune modulator.

20. The vaccine of claim 14, wherein the tumor cell is obtained from biopsy specimen.

21. The vaccine of claim 14, wherein the antigens are identified using a specific antibody or a monoclonal antibody.

22. The vaccine of claim 14, wherein the tumor cell is small cell lung cancer cell.

23. The vaccine of claim 22, wherein the antigens conjugated to a carrier are GM2, fucosyl GM1, globo H and N-propionylated polysialic acid.

24. The vaccine of claim 23, wherein the antigens are conjugated to keyhole limpet hemocyanin.

25. The vaccine of claim 24, further comprising an adjuvant, QS-21 or GPI-0100.

26. A method of treating small cell lung cancer, comprising administering an effective amount of the vaccine of claim 14 to a subject, wherein the antigens conjugated to the carrier are GM2, fucosyl GM1, globo H and N-propionylated polysialic acid, and wherein the carrier is keyhole limpet hemocyanin.

27. The method of claim 26, wherein the vaccine is administered with an adjuvant.

28. The method of claim 27, wherein the adjuvant is QS-21 or GPI-0100.

29. The method of claim 27, wherein the vaccine is administered intramuscularly or subcutaneously.

30. The method of claim 27, wherein the vaccine comprises 1 to 50 mcg of each antigen.

31. The method of claim 27, wherein the vaccine comprises 10-30 mcg each of GM2, fucosyl GM1 and Globo H and 3-10 mcg of N-propionylated polysialic acid.

32. The method of claim 26, wherein the vaccine comprises 1 mcg of N-propionylated polysialic acid and 3 mcg of fucosyl GM1.

33. A composition for treating small cell lung cancer, said composition comprising an effective amount of antigens comprising GM2, fucosyl GM1, globo H and N-propionylated polysialic acid, wherein the antigens are conjugated to keyhole limpet hemocyanin, wherein an antibody against one antigen does not inhibit other antibodies against other antigens, and wherein antibodies against the antigens have high cell surface reactivity.

34. The composition of any one of claims 33, further comprising an adjuvant, QS-21 or GPI-0100.
